(19) United States
(12) Patent Application Publication
(10) Pub. No.: US 2022/0354383 A1
(43) Pub. Date: Nov. 10, 2022

(54) BREATH SAMPLING DEVICE

(71) Applicant: NATIONAL UNIVERSITY OF SINGAPORE, Singapore (SG)

(72) Inventors: Zhunan JIA, Singapore (SG); Venky Thirumalai VENKATESAN, Singapore (SG); Fang DU, Singapore (SG)

(21) Appl. No.: 17/622,615

(22) PCT Filed: Jun. 25, 2020
(86) PCT No.: PCT/SG2020/050361

§ 371 Date: Dec. 23, 2021

(30) Foreign Application Priority Data
Jun. 25, 2019 (SG) 1020190589R

(51) Int. Cl. A61B 5/097; A61B 5/083; A61B 5/087; A61B 5/00

(52) U.S. Cl. CPC A61B 5/097; A61B 5/0836; A61B 5/087; A61B 5/486; A61B 5/742; A61B 2090/0813

(57) ABSTRACT

Disclosed is a breath sampling device comprising a buffer tube, sensor module and breath sampling port. The buffer tube has a proximal end into which a user exhales and a distal end opposite the proximal end, the sensor module is at the distal end and used for measuring parameters of a breath exhaled by the user into the buffer tube, and the breath sampling port is disposed between the proximal end and distal end. The used exhales into the distal end and a portion of the exhaled breath can be diverted into the sampling port substantially without contacting the sensor module.

### TECHNICAL FIELD

The present invention relates, in general terms, to a breath sampling device used in the analysis of exhaled breath. The present invention relates, in particular, to breath sampling devices through which a user exhales, for directing the exhaled breath into, for example, an analysis instrument or collection vessel.

### BACKGROUND

Human breath contains thousands of volatile organic compounds (VOCs) present at trace levels. These VOCs are constantly produced during metabolism and released via blood-gas exchange from the alveoli into breath. Disease is often accompanied by altered metabolic pathways and thus should result in detectable changes in VOC profile of exhaled breath. Thus, analysis of exhaled breath can facilitate identification of disease biomarkers and the formation of preliminary diagnoses of medical conditions.

Breath sampling and analysis is, as yet, an under-developed technology that has not been widely adopted or commercialised. This is generally due to the concentration of VOCs in human breath being very low. It is easy for samples to be contaminated by environmental and other confounding factors—i.e. factors that affect the accuracy of useability of results. The presence of some VOCs in exhaled breath, or the concentration of those VOCs, can also rely heavily on the rate or speed of exhalation.

In addition, some VOCs are only present, or are present in detectable or analytically relevant quantities, in particular breathing phases. For example, VOCs emitted from the oral cavity upper airways are detectable early in the exhaled breath, whereas VOCs from emitted by the alveoli are detectable later in the exhaled breath.

It is therefore difficult to ensure that a collected portion of exhaled breath is suitable for the analysis intended to be performed on that breath.

It would be desirable to overcome or ameliorate at least one of the above-described problems, or at least to provide a useful alternative.

### SUMMARY

Disclosed herein is a breath sampling device comprising:

a buffer tube having a proximal end into which a user exhales and a distal end opposite the proximal end;

a sensor module at the distal end, for measuring parameters of a breath exhaled by the user into the buffer tube; and

a breath sampling port, disposed between the proximal end and distal end, through which a portion of the exhaled breath can be diverted substantially without contacting the sensor module.

The breath sampling device may comprise a sterilisation device for sterilising an internal region of the breath sampling device. The sensor module may comprise the sterilisation module and the internal region. The sterilisation device may be an ultraviolet (UV) light source.

The buffer tube may comprise a heating jacket, the heating jacket heating the buffer tube to reduce attachment of volatile organic compounds (VOCs) to the buffer tube.

The sensor module may comprise a flow meter for sensing a flow rate of exhalation of the exhaled breath, and the breath sampling device may further comprise an output device for receiving a measurement of the flow rate from the flow meter and, if the measurement is outside an acceptable flow rate range, outputting a signal to the user to modify the flow rate to bring it into the acceptable flow rate range.

The breath sampling device may further comprise a valve for selectively opening and closing the breath sampling port, and the sensor module may comprise a carbon dioxide (CO₂) sensor for detecting an instantaneous carbon dioxide level in the exhaled breath, the sensor module determining a breathing phase from the instantaneous carbon dioxide level and actuating the valve depending on a breathing phase desired to be sampled by the breath sampling device (the desired breathing phase).

The sensor module may comprise a flow meter for sensing a flow rate of exhalation of the exhaled breath, and the breath sampling device may further comprise an output device for receiving a measurement of the flow rate from the flow meter and, if the measurement is outside an acceptable flow rate range, outputting a signal to the user to modify the flow rate to bring it into the acceptable flow rate range. The sensor module may actuate the valve only if the flow rate is within the acceptable flow rate range.

The breath sampling device may comprise a clean air supply connected to the buffer tube, for delivering clean air to the user for inhalation. The clean air supply may comprise a first one-way valve enabling passage of clean air from the clean air supply to the user in a first direction, and precluding air flow in a second direction opposite the first direction. The first one-way valve may be actuated to prevent delivery of clean air during exhalation. The sensor module may comprise a flow meter for sensing a flow rate of exhalation of the exhaled breath, the sensor module actuating the first one-way valve to prevent delivery of clean air upon the flow rate indicating commencement of exhalation and actuating the second one-way valve to prevent passage of ambient air into the buffer tube during inhalation.

The breath sampling device may further comprise a second one-way valve disposed in the buffer tube proximally of the sensor module, enabling the exhaled breath to flow distally through the sensor module in a first direction and precluding flow of air through the second one-way valve in a second direction opposite the first direction. The second one-way valve may be positioned distally of the clean air supply to prevent passage of air through the second one-way valve during inhalation of clean air.

The proximal end of the buffer tube may be adapted to engage a mouthpiece through which the user exhales into the buffer tube. The mouthpiece may be formed from a material comprising at least one of Teflon®, glass and a silanized material.

The breath sampling device may be useable to detect the presence of one or more volatile organic compounds (VOCs) in exhaled breath, and the buffer tube may comprise a removable inner tube formed from a material that is inert with respect to the VOCs. The buffer tube may be formed form a material comprising at least one of Teflon®, glass and a silanized material.

The sampling port may be attachable to at least one of a breath collection vessel and an analytical instrument. The sampling port may instead form a fixed connection for connecting to an external device such as a breath collection vessel or an analytical instrument. The breath sampling device may further comprise an adaptor for interfacing between the fixed connection and the external device.

In the present context the phrase “substantially without contacting the sensor module” means that any VOCs emitted by the sensor modules and entrained or otherwise captured in the portion of exhaled breath being diverted through the breath sampling port are in insufficient quantities to confound the results of analysis performed on that portion of exhaled breath.

In some embodiments, the buffer tube includes a removable inner tube formed from a material that is inert with respect to the VOCs being sampled. That is not to say that the inner tube, or any other feature described herein as being inert, is inert with respect to all substances.

### BRIEF DESCRIPTION OF THE DRAWINGS

Embodiments of the present invention will now be described, by way of non-limiting example, with reference to the drawings in which:

FIG. 1 is a schematic view of a breath sampling device in accordance with present teachings;

FIG. 2 is a schematic view of another embodiment of a breath sampling (i.e. collection) device in accordance with present teachings, for online breath sampling; and

FIG. 3 is a schematic view of another embodiment of a breath sampling device in accordance with present teachings, for offline breath sampling.

### DETAILED DESCRIPTION

Breath sampling devices described herein are also known as breath collection devices. Embodiments of the present breath collection devices taught herein enable collection of a desired portion of an exhaled breath while avoiding various confounding effects.

Confounding Effects

More than 1000 VOCs have been detected in human breath and the majority of these have exogenous origins. As a consequence, the contribution of VOCs from the ambient environment can have a confounding effect on the results of analysis of exhaled breath.

VOCs are also emitted from different parts of the respiratory system. Some VOCs are emitted from the upper airway or oral cavity. Other VOCs are emitted from alveoli in the lungs. An exhaled breath is therefore generally considered to have three phases-Phases 1 and 2 are air from the dead space in the oral cavity and upper airway, and Phase 3 is alveolar air from deep within the lungs. In addition, the phases can be further decomposed—for example, the term “end-tidal” breath refers to the portion of the exhaled alveolar air near to the end of Phase 3 of one exhalation.

To ensure the VOCs are present in measurable quantities, it is important to capture or sample that portion of an exhaled breath that corresponds to the VOCs being identified. Capturing breath from phases other than the desired phase can have a confounding effect on results—for example, the air from the incorrect phase may have a diluting effect.

Expiratory flow rate and hyperventilation also affect the concentration and presence of particular VOCs in exhaled breath. When asked to provide a breath sample some users also held their breath which further impacts the accuracy of results. In addition, the size of the mouthpiece into which the user blew during analysis created further changes in VOC profile.

The levels of VOCs, and the relative levels of VOCs, was therefore affected by changes in respiratory flow rate, breath holding and dimensions of the test mouthpiece.

A further confounding factor is contamination from the sample collection equipment. Given the levels of VOCs in breath are trace levels and thus very small, further contributions of very small levels of VOCs from sample collection equipment, particularly sensors used to measure breath parameters such as flow rate, can significantly influence the outcomes of analysis.

The present breath sampling devices were developed in view of the foregoing, and other, confounding factors.

Breath Collection Device

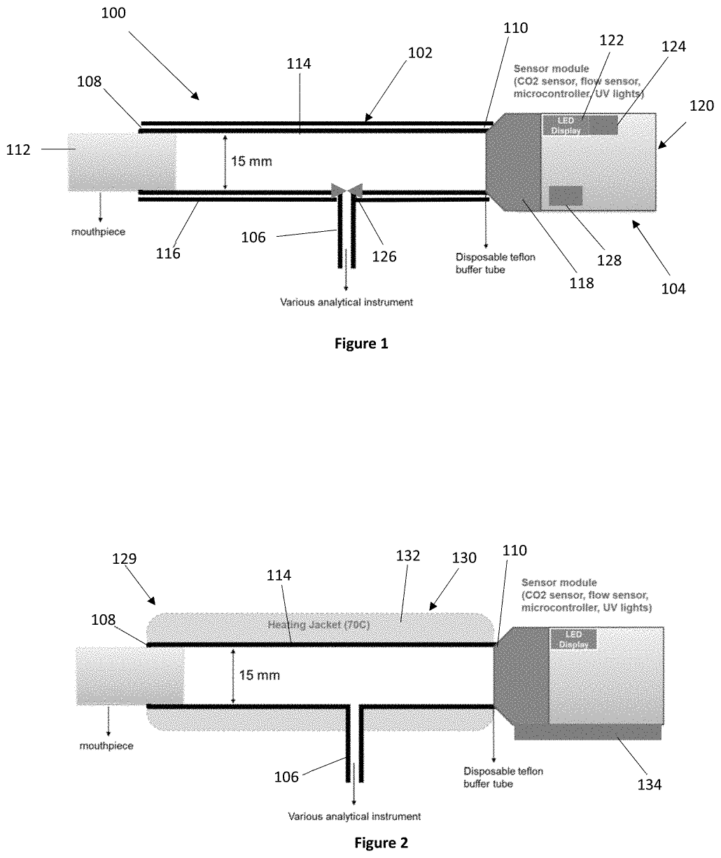

In FIG. 1, a breath sampling device 100 broadly comprises:
a buffer tube 102 into which the user exhales;
a sensor module 104; and
a breath sampling port 106.

The breath sampling device 100 may, for some embodiments, be referred to as a breath collection device where, for example, the sampling port 106 is used for collection of breath. In either case, the breath sampling device 100 is used for controlling the sampling of breath to enable a particular portion of that breath to be analysed for VOCs. The device 100 is thus useable to detect (i.e. in the detection of) the presence of one or more volatile organic compounds (VOCs) in exhaled breath

Breath is exhaled into the buffer tube 102. The buffer tube 102 has a volume sufficient to hold a small volume of exhaled air—e.g. 60 mL. Buffering a small portion of the exhaled breath increases the sampling time and improves the quality of signals or measurements derived from the portion of the exhaled breath being analysed.

The buffer tube 102 may be any desired shape. Presently, the buffer tube 102 is a cylindrical tube formed from at least one of glass, Teflon, silanized materials such as various metal oxides and glass, or any other inert material having very low VOC emission levels.

The buffer tube 102 has a proximal end 108 into which the user exhales, and a distal end 110 that is opposite to the proximal end 108. The proximal end 108 of the buffer tube 102 is adapted to engage a mouthpiece 112. While, in some embodiments, the user may exhale directly into the buffer tube 102, in the present embodiment the user exhales into the mouthpiece 112 and thereby into the buffer tube 102. The mouthpiece 112 may be detachable from the buffer tube 102 to enable independent sterilisation of the mouthpiece 112, or to allow for single-use mouthpieces.

The mouthpiece 112 may be formed from any appropriate material. For example, the mouthpiece 112, as with other components that come into contact with breath, may be formed from, for example, Teflon®, glass or a silanized material such as a silanized metal oxide.

The mouthpiece 112 may further be a one-way mouthpiece. For example, the mouthpiece 112 may comprise a one-way valve opened by blowing in one direction through the mouthpiece 112 but that cannot be opened when blowing in the opposite direction. Such valves will be understood by the skilled person in light of the present disclosure.

The buffer tube 102 comprises a removable inner tube 114 formed from a material that is inert with respect to the VOCs. Such materials are mentioned above. The inner tube 114 may have any desired diameter and, in the present embodiments, has a diameter of approximately 15 mm. The removable inner tube 114 is received in an outer sleeve 116. The inner tube 114 may engage an internal surface of the outer sleeve in a known manner—e.g. by being keyed and abutting a stop at the distal and of the buffer tube 102—such that its orientation in, and position along, the sleeve 116 is fixed when in use.

The mouthpiece 112 and inner tube 114 may be disposable so that a sterile mouthpiece 112 and inner tube 114 may be used for each sample or analysis.

At the distal end 110 of the buffer tube 102 is the sensor module 104—in the present context the sensor module being disposed “between the proximal end and distal end” includes the sensor module 104 being located at the distal end 110 and at another point between the distal end 110 and sampling port 106. The sensor module 104 may contain sensors capable of detecting the presence of particular VOCs in exhaled breath, the present sensor module 104 instead measures parameters to ensure proper operation of the device 100. The sensor module 104 is located at the distal end 110 of the buffer tube 102 and is used for measuring parameters of a breath exhaled by the user into the buffer tube 102.

As with the mouthpiece 112 and inner tube 114, to reduce the likelihood of contamination a sample by residue from a previous sample or other source, the device 100 comprises a sterilisation device 118. The sterilisation device is used for sterilising an internal region of the breath sampling device and, in the present embodiment, is part of the sensor module 104. The “internal region” may be the internal surface of the buffer tube and/or sensor module (the internal surface of the sensor module may be part of the internal surface of the buffer tube), and other internal components that will be exposed to exhaled breath.

In the present embodiment, the sterilisation device 118 thus sterilises an internal region of the sensor module 104 to ensure VOCs and other contaminants remaining after a previous sample, or gathering during a period of non-use, are removed before the next sample or analysis is conducted. A breath thus passes from a sterile mouthpiece 112 through a sterile inner tube 114 and sterilised sensor module 118.

The sterilisation device 118 presently comprises an ultraviolet (UV) light source. The sensor module 118 is also hollow with an open distal end 120. Portions of an exhaled breath that are not desired to be sampled are vented through the open distal end 120.

Disposed between the proximal end 108 and distal end 110 is the breath sampling port 106. A portion of the exhaled breath can be diverted into the breath sampling port 106 substantially without contacting the sensor module 104. The sampling port 106 may direct breath into an analytical instrument or may be attachable to a breath collection vessel. The sampling port 106 may comprise the female portion of a connector, the female portion terminating at the buffer tube 102, so that a sterile male portion from the breath collection device or analytical instrument covers the internal surface of the sampling port 106, thereby maintaining a sterile path for exhaled breath throughout the device 100. Alternatively, a disposable or cleanable bacterial filter may be place at the sampling port to maintain sterility thereof.

By positioning the sensor module 104 at the opposite end of the buffer tube 102 to the mouthpiece 112 (i.e. the proximal end 108), with the breath sampling port 106 between the proximal end 108 and sensor module 106, any VOCs emitted by the sensor module 106 are downstream of the sampling port 112. VOCs emitted from the sensor module 106 therefore substantially avoid being sampled—in this sense “substantially avoid” means they may be entirely excluded from the portion of the breath directed into the sampling tube, or may be excluded to the extent that they do not adversely affect the outcomes of analysis. Thus, the abovementioned confounding effects arising from VOCs emitted from sensors in the sensor module 104 is substantially avoided.

In previous devices, particular sensory devices have been located at the proximal end of a breath collection device. This is intuitive since the measurements are taken as soon as breath begins to be exhaled, which is believed to make the reading more accurate. As mentioned above, however, sensors can themselves emit VOCs. Advantageously, in addition to avoid entrainment or capture of VOCs from the sensor module in samples, locating the sensor module at an opposite end of the buffer tube to the end into which breath is exhaled, and downstream (i.e. distally) of the sampling port, has been found not to adversely affect the measurements taken by the sensors.

The breath sampling device 100 further comprises an output device 122. The output device 122 provides an output to the user to guide the user through exhalation manoeuvres. Particularly, the output device 122 provides the user an indication of whether their exhalation flow rate is within an acceptable range. As mentioned above, the exhalation rate and changes therein can affect the concentration of particular VOCs in an exhaled breath. Therefore, controlling exhalation rate removes a further confounding effect.

The output device 122 may comprise one or more light-emitting diodes (LEDs) or organic LEDs, a display on which a message or indicator is displayed, a haptic feedback system or any other desired output.

The output device 122 is connected to the sensor module 104. The sensor module 104 comprises a flow meter 124 for sensing a flow rate of exhalation of the exhaled breath. The output device 122 receives a measurement of the flow rate from the flow meter 124. As mentioned above, if the measurement is outside an acceptable flow rate range, the output device 122 will output a signal to the user to modify the flow rate to bring it into the acceptable flow rate range.

In one embodiment, the output device comprises a red LED and a green LED. The green LED is ON if the flow rate is within the acceptable range and is OFF if the flow rate is outside the acceptable range. Similarly, the red LED is ON if the flow rate is outside the acceptable range and is OFF if the flow rate is within the acceptable range. The output may alternatively comprise two LEDs of different colours, one of which indicates the flow rate is too high and the other of which indicates the flow rate is too low.

The device 100 may further comprise a power supply (e.g. a battery—not shown) for powering the flow meter 124 and/or other components of the sensor module 104.

The breath sampling device 100 further includes a valve 126 for selectively opening and closing the breath sampling port 106. In some embodiments, no valve may be necessary. In the present embodiment, however, the sensor module 104 includes a carbon dioxide (CO₂) sensor 128 for detecting an instantaneous carbon dioxide level (e.g. CO₂ partial pressure) in the exhaled breath. The sensor module 104 determines a breathing phase from the instantaneous carbon dioxide level and actuates the valve 126 depending on a breathing phase desired to be sampled by the breath sampling device 100 (the desired breathing phase). In other embodiments, the output device 122 receives an output from the sensor module 104 indicating that the desired breathing phase has been reached, and outputs a signal to the user to instruct the user to manually switch the valve 126 to cause breath to be directed through the sampling port 106.

To avoid capturing samples where the exhalation rate is outside the acceptable range, where the valve 126 is operated by the sensor module 104, the sensor module 104 may only actuate the valve 126 if the flow rate is within the acceptable flow rate range. Similarly, where the valve is manually operated, the output device 122 may only instruct the user to operate the valve 126 if the desired flow rate is maintained.

Online Sampling

Online sampling refers to sampling where the sample of the exhaled breath is analysed upon being sampled. In general, this will be applied in a hospital or clinical setting. It was found that VOCs stick to internal surfaces of the buffer tube due to temperature and humidity of the exhaled breath.

A breath sampling device 129 for online sampling is shown in FIG. 2. The breath sampling device 129 includes a buffer tube 130. The buffer tube 130 is similar to buffer tube 102 but also includes a heating jacket 132. The heating jacket 132 heats the inner tube 114, reducing or preventing VOC attachment. This may also reduce or prevent moisture attachment to the inner tube 114. Moreover, this may assist in providing a more consistent sampling temperature and humidity for longitudinal studies. Using the heating jacket 132 thus removes a further confounding effect, namely that arising from temperature and humidity variations.

Moreover, as mentioned above, previous devices have included sensors near the end into which the user exhales. For the heating jacket to prevent VOC attachment to the buffer tube, the heating jacket should extend along the buffer tube from the proximal end 108 to the sampling port 106. Additionally, the heating jacket should be heated to around 65° C. to 75° C., and preferably around 70° C. Such temperatures affect measurements taken by sensors and would thus affect sensors mounted near the proximal end of the buffer tube. By positioning the sensor module 104, and thus the sensors, at the distal end 110, and distally of the sampling port 106, the heat from the heating jacket 132 does not affect sensor measurements.

The heating jacket 132 may form the outer sleeve of the buffer tube 130, as shown, and therefore be similar in operation to outer sleeve 116 of buffer tube 102. In other embodiments, the heating jacket 132 may be received around the outer sleeve.

The breath sampling device 129 further comprises an environment sampler 134. The environment sampler 134 measures VOCs present in the ambient environment that the user may be expected to have inhaled. The presence of those VOCs can therefore be accounted for when assessing VOC levels in the portion of breath being analysed. This helps remove the confounding effect of environmental VOCs.

The environment sampler 134 may further include temperature and humidity sensors and other sensors as desired to improve the accuracy of analysis of the portion of the exhaled breath being sampled. In other embodiments, the environment sampler may be an external device in communication with the analytical instrument.

Notably, in the online sampling device the valve 126 may not be required—the present embodiment thus excludes the valve 126. Instead, upon the CO₂ sensor identifying that the desired portion of the exhaled breath has been reached, the analytical instrument (i.e. external device) draws air from the buffer tube 130. When the CO₂ determines that the desired phase has ended, the analytical instrument no longer draws breath in from the buffer tube 130.

Offline Sampling

Offline sampling involves collection of breath. Typically, a breath bag or collection vessel is connected to a sampling port and breath is directed into the bag or vessel. The bag or vessel is then sealed and sent for analysis. Offline sampling devices are therefore preferred for in-home use.

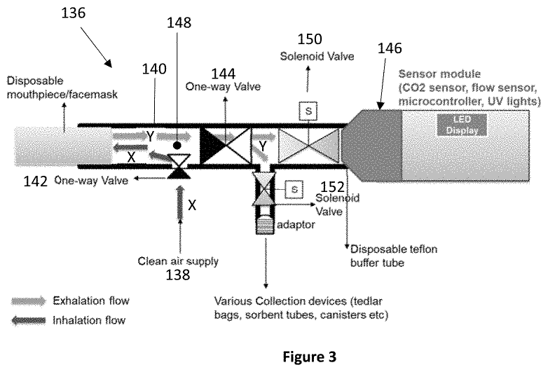

FIG. 3 shows an offline breath sampling device 136.

One of the main confounding factors for offline analysis is environmental VOCs. To reduce the impact of environmental VOCs, the breath sampling device 136 comprises a clean air supply 138 connected to the buffer tube 140. The clean air supply 138 delivers clean air to the user for inhalation. Presently, the clean air supply 138 supplies clean air into the buffer tube 140.

To ensure clean air passes from the clean air supply 138 into the buffer tube 140 but not from the buffer tube 140 into the clean air supply 138, the clean air supply 138 comprises a one-way valve 142. The one-way valve 142 will herein be referred to as a “first” one-way valve 142. The first one-way valve 142 enables passage of clean air from the clean air supply 138 to the user in a first direction marked by arrows X, and precludes air flow in a second direction opposite the first direction. Arrows Y illustrate the passage of air along the buffer tube 140 as would be the case during exhalation, and that the exhaled air does not enter the clean air supply 138.

The first one-way valve 142 may be passive, and thus operate based on the difference in pressure in the buffer tube 140 between the pressure during inhalation and that during exhalation. Alternatively, the first one-way valve 142 may be actuated (e.g. isolated to prevent flow in either direction) to prevent delivery of clean air during exhalation.

In non-passive embodiments, the first one-way valve 142 may be manually operated.

The breath sampling device 136 also includes a second one-way valve 144. The second one-way valve 144 is disposed in the buffer tube 140 proximally of the sensor module 146. The second one-way valve 144 enables the exhaled breath to flow distally through the sensor module 146 in a first direction and precludes flow of air through the second one-way valve 144 in a second direction opposite the first direction.

The second one-way valve 144 is positioned distally of the clean air supply 138. Thus, during inhalation the internal volume of the buffer tube 140 proximal of the second one-way valve 144 (volume 148) is flooded with clean air drawn from the clean air supply 138. During inhalation, the second one-way valve 144 prevents ambient air from flowing through the sensor module 146 into the proximal internal volume 148 of the buffer tube 140.

The offline sampling device 136 also includes a first valve 150 and a second valve 152 (see also valve 126 of FIG. 1). Operation of the valves 150, 152 is controlled by the sensor module 146. Valves 150, 152 will hereinafter be referred to as solenoid valves though any other valve controllable by the sensor module 146 may be used. When the CO₂ sensor of the sensor module 146 indicates the desired breathing phase has been reached, the sensor module 146 closes solenoid valve 150 to prevent flow of air through the sensor module 146, and opens solenoid valve 152 to allow the desired portion of the exhaled breath to flow into a sample collection device such as a bag or breath capture vessel. Until the desired breath portion has been reached, valve 150 will be open and valve 152 closed. In some embodiments, the CO₂ sensor may be placed between the sampling port and valve 152, so that valve 152 can be reopened after the desired breath portion has passed. Alternatively, valve 152 may be omitted —in such embodiments, when the CO₂ pressure (e.g. partial pressure) is below or not equal to a desired value or range—indicating a particular breath phase-valve 152 will remain closed, when the desired value is reached valve 152 will be opened and breath will naturally flow both through the sensor module 146 and through the sampling port, and once the CO₂ level is no longer present (i.e. the desired breath phase has passed) valve 152 is closed.

The offline device 136 may further comprise an environment sampler similar to environment sampler 134 of FIG. 2.

It will be appreciated that many further modifications and permutations of various aspects of the described embodiments are possible. Accordingly, the described aspects are intended to embrace all such alterations, modifications, and variations that fall within the spirit and scope of any appended claims.

Throughout this specification and any claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A breath sampling device comprising:
a buffer tube having a proximal end into which a user exhales and a distal end opposite the proximal end;
a sensor module at the distal end, for measuring parameters of a breath exhaled by the user into the buffer tube; and
a breath sampling port, disposed between the proximal end and distal end, through which a portion of the exhaled breath can be diverted substantially without contacting the sensor module.

2. A breath sampling device according to claim 1, comprising a sterilisation device for sterilising an internal region of the breath sampling device.

3. A breath sampling device according to claim 2, wherein the sensor module comprises the sterilisation device and the internal region.

4. A breath sampling device according to claim 2 or 3, wherein the sterilisation device is an ultraviolet (UV) light source.

5. A breath sampling device according to claim 1, wherein the buffer tube comprises a heating jacket, the heating jacket heating the buffer tube to reduce attachment of volatile organic compounds (VOCs) to the buffer tube.

6. A breath sampling device according to claim 1, wherein the sensor module comprises a flow meter for sensing a flow rate of exhalation of the exhaled breath, and the breath sampling device further comprises an output device for receiving a measurement of the flow rate from the flow meter and, if the measurement is outside an acceptable flow rate range, outputting a signal to the user to modify the flow rate to bring it into the acceptable flow rate range.

7. A breath sampling device according to claim 1, further comprising a valve for selectively opening and closing the breath sampling port, wherein the sensor module comprises a carbon dioxide (CO₂) sensor for detecting an instantaneous carbon dioxide level in the exhaled breath, and the sensor module determines a breathing phase from the instantaneous carbon dioxide level and actuates the valve depending on a breathing phase desired to be sampled by the breath sampling device (the desired breathing phase).

8. A breath sampling device according to claim 6, wherein the sensor module comprises a flow meter for sensing a flow rate of exhalation of the exhaled breath, and the breath sampling device further comprises an output device for receiving a measurement of the flow rate from the flow meter and, if the measurement is outside an acceptable flow rate range, outputting a signal to the user to modify the flow rate to bring it into the acceptable flow rate range.

9. A breath sampling device according to claim 8, wherein the sensor module actuates the valve only if the flow rate is within the acceptable flow rate range.

10. A breath sampling device according to claim 6, wherein the output device comprises a light-emitting diode (LED) display or organic LED display for providing an output to the user to guide the user through exhalation manoeuvres.

11. A breath sampling device according to claim 1, further comprising a clean air supply connected to the buffer tube, for delivering clean air to the user for inhalation.

12. A breath sampling device according to claim 11, wherein the clean air supply comprises a first one-way valve enabling passage of clean air from the clean air supply to the user in a first direction, and precluding air flow in a second direction opposite the first direction.

13. A breath sampling device according to claim 12, wherein the first one-way valve can be actuated to prevent delivery of clean air during exhalation.

14. A breath sampling device according to claim 12, further comprising a second one-way valve disposed in the buffer tube proximally of the sensor module, enabling the exhaled breath to flow distally through the sensor module in a first direction and precluding flow of air through the second one-way valve in a second direction opposite the first direction.

15. A breath sampling device according to claim 14, wherein the second one-way valve is positioned distally of the clean air supply to prevent passage of air through the second one-way valve during inhalation of clean air.

16. A breath sampling device according to claim 1, wherein the proximal end of the buffer tube is adapted to engage a mouthpiece through which the user exhales into the buffer tube.

17. A breath sampling device according to claim 16, wherein the mouthpiece is formed from a material comprising at least one of Teflon®, glass and a silanized material.

18. A breath sampling device according to claim 1, being useable to detect the presence of one or more volatile organic compounds (VOCs) in exhaled breath, wherein the buffer tube comprises a removable inner tube formed from a material that is inert with respect to the VOCs.

19. A breath sampling device according to claim 18, wherein the buffer tube is formed form a material comprising at least one of Teflon®, glass and a silanized material.

20. A breath sampling device according to claim 1, wherein the sampling port is attachable to at least one of a breath collection vessel and an analytical instrument.
